(19) United States
(12) Patent Application Publication
(10) Pub. No.: US 2007/0111989 A1
(43) Pub. Date: May 17, 2007

(54) Novel diazabicyclononene derivatives and use

(76) Inventors: Olivier Bezencon, Riehen (CH); Daniel Bur, Therwil (CH); Walter Fischli, Allschwil (CH); Lubos Remen, Allschwil (CH); Sylvia Richard-Bildstein, Dietwiller (FR); Thierry Sifferlen, Guewenheim (FR); Thomas Weller, Binningen (CH)

Correspondence Address: HOXIE & TSO LLP, 374 MILLBURN AVENUE, SUITE 300 E, MILLBURN, NJ, 07041, US

(21) Appl. No.: 10/581,829

(22) PCT Filed: Nov. 30, 2004
(86) PCT No.: PCT/EP04/13578

§ 371 Date: Jun. 2, 2006

(51) Int. Cl. A61K 31/551; C07D 487/04

(52) U.S. Cl. 514/215; 514/221; 540/581; 540/472

(57) ABSTRACT

The invention relates to novel 3,9-diazabicyclo[3.3.1]nonene derivatives of formula (I), wherein Z is 0 or 1, one of m, n is 0 and the other is 1, and their use as inhibitors of renin.

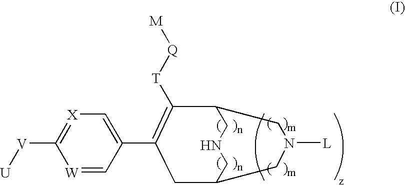

The invention relates to novel five-membered heteroaryl derivatives of the general formula (I). The invention also concerns related aspects including processes for the preparation of the compounds, pharmaceutical compositions containing one or more compounds of formula (I) and especially their use as renin inhibitors in cardiovascular events and renal insufficiency.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J Hypertens., 1993, 11, 1155).

In patients inhibition of ACE thus leads to bradykinin accumulation causing cough (5-20%) and potentially life-threatening angioneurotic edema (0.1-0.2%) (Israili Z. H. et al, Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

Only limited clinical experience (Azizi M. et al., J Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, renin inhibitors with good oral bioavailability and long duration of action are required. Recently, the first non-peptide renin inhibitors were described which show high in vitro activity (Oefner C. et al., Chem. Biol., 1999, 6, 127; Patent Application WO97/09311; Märki H. P. et al., Il Farmaco, 2001, 56, 21). However, the development status of these compounds is not known.

The present invention relates to the identification of renin inhibitors of a non-peptidic nature and of low molecular weight. Described are orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis. So, the present invention describes these non-peptidic renin inhibitors.

The present invention describes non-peptidic renin inhibitors.

In particular, the present invention relates to novel compounds of the general formula I, 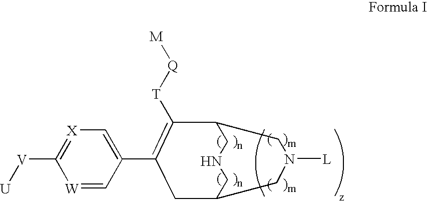
wherein
X and W represent independently a nitrogen atom or a —CH— group;
V represents —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; or —CH₂—CH₂-A-CH₂—CH₂—B—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; lower alkenylene;
M represents aryl-O(CH₂)ᵥR⁷; heteroaryl-O(CH₂)ᵥR⁷; aryl-O(CH₂)ᵥO(CH₂)_{w}R⁷; heteroaryl-(CH₂)ᵥO(CH₂)_{w}R⁷; aryl-OCH₂CH(R⁶)CH₂R⁵; heteroaryl-OCH₂CH(R⁶)CH₂R⁵;
L represents —R³; —COR³; —COOR³; —CONR²R³; —SO₂R³; —SO₂NR²R³; —COCH(Aryl)₂;
R¹ represents hydrogen; lower alkyl; lower alkenyl; lower alkinyl; cycloalkyl; aryl; cycloalkyl-lower alkyl;
R² and R²′ independently represent hydrogen; lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, CO-morpholin-4-yl, CO—((4-loweralkyl)piperazin-1-yl), —NH(NH)NH₂, —NR⁴R⁴′ or lower alkyl, with the proviso that a carbon atom is attached at the most to one heteroatom in case this carbon atom is sp3-hybridized;
R⁴ and R⁴′ independently represent hydrogen; lower alkyl; cycloalkyl; cycloalkyl-lower alkyl; hydroxy-lower alkyl; —COOR²; —CONH₂;
R⁵ represents —OH, lower alkoxy, —OCOR², —COOR², —NR²R²′, —OCONR²R²′, —NCONR²R²′, cyano, —CONR²R²′, SO₃H, —SONR²R²′, —CO-morpholin-4-yl, —CO—((4-loweralkyl)piperazin-1-yl), —NH(NH)NH₂, —NR⁴R⁴′, with the proviso that a carbon atom is attached at the most to one heteroatom in case this carbon atom is sp3-hybridized;
R⁶ represents —OH, OR²; OCOR²; OCOOR²; or R⁶ and R⁵ form together with the carbon atoms to which they are attached a 1,3-dioxolane ring which is substituted in position 2 with R² and R²′; or R⁶ and R⁵ form together with the carbon atoms to which they are attached a 1,3-dioxolan-2-one ring;
R⁷ represents lower alkoxy;
m and n represent the integer 0 or 1, with the proviso that in case m represents the integer 1, n is the integer 0, and in case n represents the integer 1, m is the integer 0;
p is the integer 1, 2, 3 or 4;
r is the integer 3, 4, 5, or 6;
s is the integer 2, 3, 4, or 5;
t is the integer 1, 2, 3, or 4;
u is the integer 1, 2, or 3;
v is the integer 1, 2, 3, or 4;
w is the integer 1 or 2;
z is the integer 0 or 1
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

In the definitions of general formula I—if not otherwise stated—the term lower alkyl, alone or in combination with other groups, means saturated, straight and branched chain groups with one to seven carbon atoms, preferably one to four carbon atoms that can be optionally substituted by halogens. Examples of lower alkyl groups are methyl, ethyl, n-propyl, iso-propyl, n-butyl, iso-butyl, sec-butyl, tert-butyl, pentyl, hexyl and heptyl. The methyl, ethyl nad isopropyl groups are preferred.

The term lower alkoxy refers to a R—O group, wherein R is a lower alkyl. Examples of lower alkoxy groups are methoxy, ethoxy, propoxy, iso-propoxy, iso-butoxy, sec-butoxy and tert-butoxy.

The term lower alkenyl, alone or in combination with other groups, means straight and branched chain groups comprising an olefinic bond and consisting of two to seven carbon atoms, preferably two to four carbon atoms, that can be optionally substituted by halogens. Examples of lower alkenyl are vinyl, propenyl or butenyl.

The term lower alkinyl, alone or in combination with other groups, means straight and branched chain groups comprising a triple bond and consisting of two to seven carbon atoms, preferably two to four carbon atoms, that can be optionally substituted by halogens. Examples of lower alkinyl are ethinyl, propinyl or butinyl.

The term lower alkylene, alone or in combination with other groups, means straight and branched divalent chain groups with one to seven carbon atoms, preferably one to four carbon atoms, that can be optionally substituted by halogens. Examples of lower alkylene are ethylene, propylene or butylene.

The term lower alkenylene, alone or in combination with other groups, means straight and branched divalent chain groups comprising an olefinic bond and consisting of two to seven carbon atoms, preferably two to four carbon atoms, that can be optionally substituted by halogens. Examples of lower alkenylene are vinylene, propenylene and butenylene.

The term lower alkylenedioxy, refers to a lower alkylene substituted at each end by an oxygen atom. Examples of lower alkylenedioxy groups are preferably methylenedioxy and ethylenedioxy.

The term lower alkylenoxy refers to a lower alkylene substituted at one end by an oxygen atom. Examples of lower alkylenoxy groups are preferably methylenoxy, ethylenoxy and propylenoxy.

The term halogen means fluorine, chlorine, bromine or iodine, preferably fluorine, chlorine and bromine.

The term cycloalkyl alone or in combination, means a saturated cyclic hydrocarbon ring system with 3 to 7 carbon atoms, e.g. cyclopropyl, cyclobutyl, cyclopentyl, cyclohexyl and cycloheptyl, which can be optionally mono- or multisubstituted by lower alkyl, lower alkenyl, lower alkenylene, lower alkoxy, lower alkylenoxy, lower alkylenedioxy, hydroxy, halogen, —CF₃, —NR¹R¹′, —NR¹C(O)R¹′, —NR¹S(O₂)R1′, —C(O)NR¹R¹′, lower alkylcarbonyl, —COOR¹, —SR¹, —SOR¹, —SO₂R¹, —SO₂NR¹R¹′ whereby R¹′ represents hydrogen; lower alkyl; lower alkenyl; lower alkinyl; cycloalkyl; aryl; cycloalkyl-lower alkyl. The cyclopropyl group is a preferred group.

The term aryl, alone or in combination, relates to the phenyl, the naphthyl or the indanyl group, preferably the phenyl group, which can be optionally mono- or multisubstituted by lower alkyl, lower alkenyl, lower alkinyl, lower alkenylene or lower alkylene forming with the aryl ring a five- or six-membered ring, lower alkoxy, lower alkylenedioxy, lower alkylenoxy, hydroxy, hydroxy-lower alkyl, halogen, cyano, —CF₃, —OCF₃, —NR¹R¹′, —NR¹R¹′-lower alkyl, —NR¹C(O)R¹′, —NR₁S(O₂)R¹, —C(O)NR¹R¹′, —NO₂, lower alkylcarbonyl, —COOR¹, —SR¹, —SOR¹, —SO₂R¹, —SO₂NR¹R¹′, benzyloxy, whereby R¹′ has the meaning given above. Preferred substituents are halogen, lower alkoxy, lower alkyl, CF₃, OCF₃.

The term aryloxy refers to an Ar—O group, wherein Ar is an aryl. An example of a lower aryloxy group is phenoxy.

The term heterocyclyl, alone or in combination, means saturated or unsaturated (but not aromatic) five-, six- or seven-membered rings containing one or two nitrogen, oxygen or sulfur atoms which may be the same or different and which rings can be optionally substituted with lower alkyl, hydroxy, lower alkoxy and halogen. The nitrogen atoms, if present, can be substituted by a —COOR² group. Examples of such rings are piperidinyl, morpholinyl, thiomorpholinyl, piperazinyl, tetrahydropyranyl, dihydropyranyl, 1,4-dioxanyl, pyrrolidinyl, tetrahydrofuranyl, dihydropyrrolyl, imidazolidinyl, dihydropyrazolyl, pyrazolidinyl, dihydroquinolinyl, tetrahydroquinolinyl, tetrahydroisoquinolinyl.

The term heteroaryl, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing one oxygen and one nitrogen atom and benzofused derivatives thereof; five-membered aromatic rings containing a sulfur and a nitrogen or an oxygen atom and benzofused derivatives thereof; five-membered aromatic rings containing two nitrogen atoms and benzofused derivatives thereof; five-membered aromatic rings containing three nitrogen atoms and benzofused derivatives thereof, or a tetrazolyl ring. Examples of such ring systems are furanyl, thiophenyl, pyrrolyl, pyridinyl, pyrimidinyl, indolyl, quinolinyl, isoquinolinyl, imidazolyl, triazinyl, thiazinyl, thiazolyl, isothiazolyl, pyridazinyl, pyrazolyl, oxazolyl, isoxazolyl, coumarinyl, benzothiophenyl, quinazolinyl, quinoxalinyl. Such rings may be adequately substituted with lower alkyl, lower alkenyl, lower alkinyl, lower alkylene, lower alkenylene, lower alkylenedioxy, lower alkyleneoxy, hydroxy-lower alkyl, lower alkoxy, hydroxy, halogen, cyano, —CF₃, —OCF₃, —NR¹R¹′, —NR¹R¹′-lower alkyl, —N(R¹)COR¹, —N(R¹)SO₂R¹, —CONR¹R¹′, —NO₂, lower alkylcarbonyl, —COOR¹, —SR¹, —SOR¹, —SO₂R¹, —SO₂NR¹R¹′, another aryl, another heteroaryl or another heterocyclyl and the like, whereby R¹′ has the meaning given above.

The term heteroaryloxy refers to a Het-O group, wherein Het is a heteroaryl.

The term cycloalkyl-lower alkyl refers to a cycloalkyl group which is substituted with a lower alkyl group as defined above.

The term aryl-lower alkyl refers to aryl group which is substituted with a lower alkyl group as defined above.

The term heteroaryl-lower alkyl refers to a heteroalkyl group which is substituted with a lower alkyl group as defined above.

The term heterocyclyl-lower alkyl refers to a heterocyclyl group which is substituted with a lower alkyl group as defined above.

The term aryloxy-lower alkyl refers to aryloxy group which is substituted with a lower alkyl group as defined above.

The term heteroaryloxy-lower alkyl refers to a heteroaryloxy group which is substituted with a lower alkyl group as defined above.

The term hydroxy-lower alkyl refers to a lower alkyl group which is substituted with a hydroxyl group.

The term lower alkylcarbonyl refers to a —CO-lower alkyl group.

The term sp3-hybridized refers to a carbon atom and means that this carbon atom forms four bonds to four substituents placed in a tetragonal fashion around this carbon atom.

The expression pharmaceutically acceptable salts encompasses either salts with inorganic acids or organic acids like hydrochloric or hydrobromic acid, sulfuric acid, phosphoric acid, citric acid, formic acid, acetic acid, maleic acid, tartaric acid, benzoic acid, methanesulfonic acid, p-toluenesulfonic acid, and the like that are non toxic to living organisms or in case the compound of formula I is acidic in nature with an inorganic base like an alkali or earth alkali base, e.g. sodium hydroxide, potassium hydroxide, calcium hydroxide and the like.

The compounds of the general formula I can contain two or more asymmetric carbon atoms and may be prepared in form of optically pure enantiomers, mixtures of enantiomers such as racemates, diastereomers, mixtures of diastereomers, diastereomeric racemates, mixtures of diastereomeric racemates, and the meso-form and pharmaceutically acceptable salts thereof.

The present invention encompasses all these forms. Mixtures may be separated in a manner known per se, i.e. by column chromatography, thin layer chromatography, HPLC or crystallization.

A group of preferred compounds are compounds of general formula I wherein X, W, V, U, T, Q, L, and M are as defined in general formula I above and wherein

z is 1

n is 0 and

m is 1.

Another group of preferred compounds of general formula I are those wherein X, W, V, U, T, Q, M, m, and n are as defined in general formula I above and

z is 1 and

L represents H; —CoR³″; —COOR³″; —CONR²″R³″;

whereby R²″ and R³″ represent independently lower alkyl, lower cycloalkyl-lower alkyl, which lower alkyl and lower cycloalkyl-lower alkyl groups are unsubstituted or monosubstituted with halogen, cyano, hydroxy, —OCOCH₃, —CONH₂, —COOH, —NH₂, with the proviso that a carbon atom is attached at the most to one heteroatom in case this carbon atom is sp3-hybridized.

Another group of preferred compounds of general formula I above are those wherein X, W, V, U, L, m, n, and z are as defined in general formula I and

T is —CONR¹—;

Q is methylene;

M is aryl-O(CH₂)ᵥR⁷; heteroaryl-O(CH₂)ᵥR⁷; aryl-OCH₂CH(R⁶)CH₂R⁵; heteroaryl-OCH₂CH(R⁶)CH₂R⁵.

Another group of even more preferred compounds of general formula I are those wherein X, W, U, L, T, Q, M, m, n, and z are as defined in general formula I above and

V is —CH₂CH₂O—; —CH₂CH₂CH₂O—; —OCH₂CH₂O—.

Another group of also more preferred compounds of general formula I are those wherein V, U, T, Q, M, L, m, n, and z are as defined in general formula I above and

X and W represent —CH—.

Another group of also more preferred compounds of general formula I are those wherein X, W, V, Q, T, M, L, m, n, and z are as defined in general formula I above and

U is a mono-, di-, or trisubstituted phenyl wherein the substituents are halogen; lower alkyl or lower alkoxy.

Most preferred compounds of general formula I are those wherein

X and W represent a —CH— group;

V represents -A-(CH₂)s-;

A represents —O—;

U represents phenyl, trisubstituted with halogen;

T represents —CONR¹—;

Q represents C1-C4 alkyl;

M represents phenyl —O—(CH₂)v R⁷ or pyridyl-O—(CH₂)v R⁷;

L represents R³;

R¹ represents cycloalkyl;

R³ represents hydrogen, C1-C4 alkyl;

R⁷ represents C1-C4 alkoxy;

m represents the integer 1;

n represents the integer 0;

z represents the integer 1;

s represents the integer 3;

v represents the integer 2

and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

Most preferred compounds of general formula I are also those wherein

X and W represent a —CH— group;

V represents —O—CH₂—CH₂—CH₂—;

U represents phenyl, trisubstituted independently with Fluoro and Chloro;

T represents —CONR¹—;

Q represents —CH₂—;

M represents phenyl —O— (CH₂)v R⁷ or pyridyl-O— (CH₂)v R⁷;

L represents R³;

R¹ represents cyclopropyl;

R³ represents hydrogen;

R⁷ represents methoxy;

m represents the integer 1;

n represents the integer 0;

z represents the integer 1;

s represents the integer 3;

v represents the integer 2;

and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

Especially preferred compounds of general formula I are those selected from the group consisting of:
(rac.)-(1R*,5S*)-7-{4-[3-(2-chloro-3,6-difluorophenoxy)propyl]phenyl}-3,9-diazabicyclo[3.3.1]non-6-ene-6-carboxylic acid cyclopropyl-[2-(2-methoxy-ethoxy)-3-methylpyridin-4-ylmethyl]amide, and
(rac.)-(1R*,5S*)-7-{4-[3-(2-chloro-3,6-difluorophenoxy)propyl]phenyl}-3,9-diazabicyclo[3.3.1]non-6-ene-6-carboxylic acid cyclopropyl-[3-(2-methoxy-ethoxy)-2-methylbenzyl]amide.

The invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, which method comprises administrating a compound as defined above to a human being or animal.

In another embodiment, the invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In another embodiment, the invention relates to a method for the treatment and/or prophylaxis of diseases, which are associated with a dysregulation of the renin-angiotensin system as well as for the treatment of the above-mentioned diseases.

The invention also relates to the use of compounds of formula (I) for the preparation of a medicament for the treatment and/or prophylaxis of the above-mentioned diseases.

A further aspect of the present invention is related to a pharmaceutical composition containing at least one compound according to general formula (I) and pharmaceutically acceptable carrier materials or adjuvants. This pharmaceutical composition may be used for the treatment or prophylaxis of the above-mentioned disorders; as well as for the preparation of a medicament for the treatment and/or prophylaxis of the above-mentioned diseases.

Derivatives of formula (I) or the above-mentioned pharmaceutical compositions are also of use in combination with other pharmacologically active compounds comprising ACE-inhibitors, neutral endopeptidase inhibitors, angiotensin II receptor antagonists, endothelin receptors antagonists, vasodilators, calcium antagonists, potassium activators, diuretics, sympatholitics, beta-adrenergic antagonists, alpha-adrenergic antagonists or with other drugs beneficial for the prevention or the treatment of the above-mentioned diseases.

In a preferred embodiment, this amount is comprised between 2 mg and 1000 mg per day.

In a particular preferred embodiment, this amount is comprised between 1 mg and 500 mg per day.

In a more particularly preferred embodiment, this amount is comprised between 5 mg and 200 mg per day.

All forms of prodrugs leading to an active component comprised by general formula (I) above are included in the present invention.

Compounds of formula (I) and their pharmaceutically acceptable acid addition salts can be used as medicaments, e.g. in the form of pharmaceutical compositions containing at least one compound of formula (I) and pharmaceutically acceptable inert carrier material or adjuvants. These pharmaceutical compositions can be used for enteral, parenteral, or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The production of pharmaceutical preparations can be effected in a manner which will be familiar to any person skilled in the art by bringing the described compounds of formula (I) and their pharmaceutically acceptable acid addition salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injections are, for example, water, alcohols, polyols, glycerols and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of compounds of formula (I) can vary within wide limits depending on the disease to be controlled, the age and the individual condition of the patient and the mode of administration, and will, of course, be fitted to the individual requirements in each particular case.

Another aspect of the invention is related to a process for the preparation of a pharmaceutical composition comprising a derivative of the general formula (I). According to said process, one or more active ingredients of the general formula (I) are mixing with inert excipients in a manner known per se.

The compounds of general formula I can be manufactured by the methods outlined below, by the methods described in the examples or by analogous methods.

Preparation of the Precursors:

Precursors are compounds which were prepared as key intermediates and/or building blocks and which were suitable for further transformations in parallel chemistry. Most of the chemistry applyable here has already been described in the patent applications WO03/093267 and WO04/002957.

As illustrated in Scheme 1 the known compound A can be derivatised into the corresponding triflate B. A Negishi-type coupling (or any other coupling catalysed by a transition metal) leads to a compound of type C wherein R^{a} represents a precursor for the fragment U—V, as defined in general formula (I). R^{a} can be easily transformed into the fragment U—V using elemental chemical operation. After protecting group manipulation (→compound of type D), adjustment of the W—V—U linker is possible for instance by deprotection and a Mitsunobu-type reaction, leading to a compound of type E. Hydrolysis of the ester leads to a carboxylic acid of type F, then an amide coupling for instance to a compound of type G. Removal of the Boc-protecting group and alkylation, or acylation, leads to a precursor of type H.

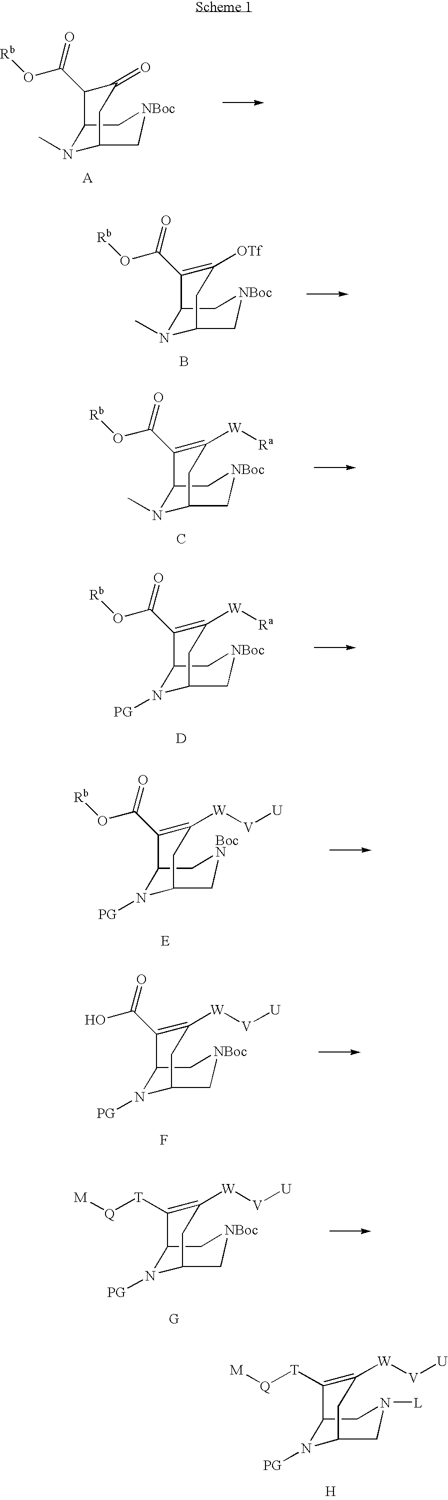

The bromoaryl components can be prepared as described in Scheme 2. A Mitsunobu coupling (→compounds of type J) or the alkylation of an alcohol with a benzylic chloride (or bromide, → compounds of type K) are often the most convenient methods. Derivatives L and M were prepared in one step from 1-(3-chloropropoxymethyl)-2-methoxybenzene (Vieira E. et al, Bioorg. Med. Chem. Letters, 1999, 9, 1397) or 3-(5-bromopyridin-2-yloxy)propan-1-ol (Patent Application WO 98/39328) according to these methods. Other methods for the preparation of ethers or thioethers, like a Williamson synthesis, can be used as well (see e.g. March, J, “Advanced Organic Chemistry,”, 3^{rd} ed., John Wiley and sons, 1985). 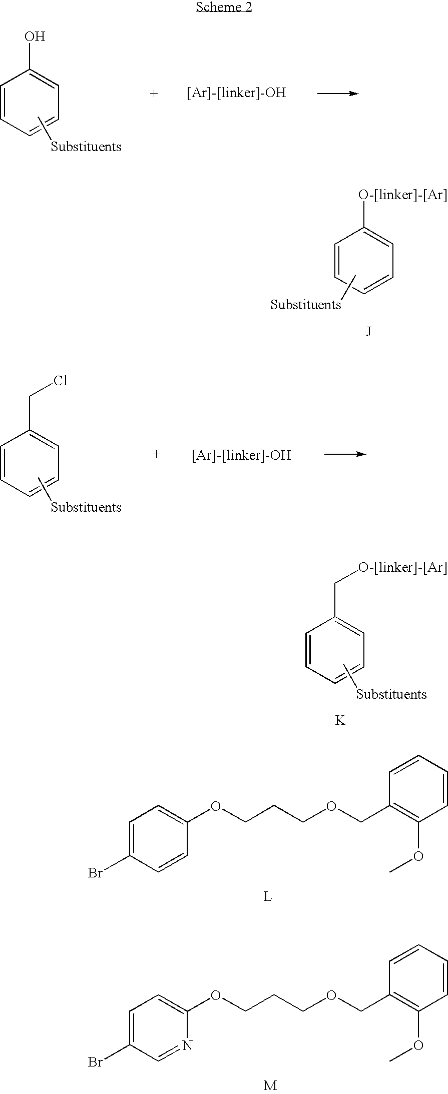
Preparation of the Secondary Amines

The secondary amines can be prepared for instance as described in Scheme 3. The pyridine derivative N can be prepared from commercially available 2-chloro-isonicotinoyl chloride. Deprotonation at the 3-position of this derivative, for instance with BuLi, and subsequent alkylation with a suitable electrophile leads to a derivative of type O, wherein R^{d} represents a suitable substituent that can be introduced by this chemistry, and can be transformed later into a desired substituent a described in general formula I. Reduction of the amide into an aldehyde with DIBAL leads to a compound of type P, then a reductive amination leads to an amine of type Q, wherein R¹ represents a substituent as defined above.

Finally substitution of the chlorine atom with an alcohol of type HO(CH2)ᵥR⁷, where as R⁷ may still be protected, leads to an amine of type R. An alcohol of type HO(CH₂)ᵥO(CH₂)_{w}R⁷ or HOCH₂CH(R⁶)CH₂R⁵ can be introduced in the same way.

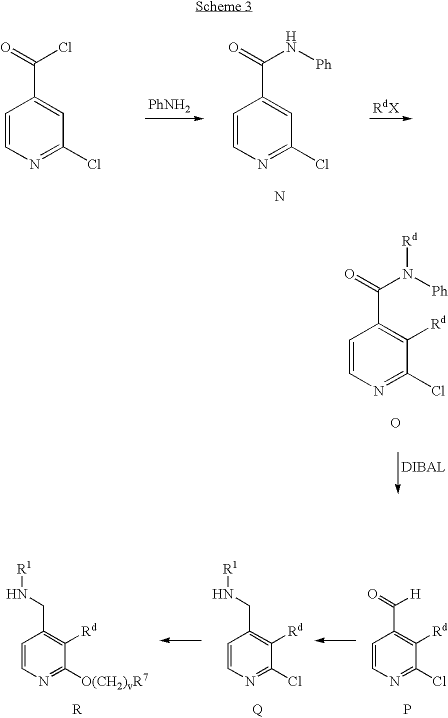

In the case of phenyl derivatives it is better to start from a compound of type S, wherein PG′ represents a suitable protecting group. Amide coupling with N-methylaniline leads to a derivative of type T, then deprotection to a derivative of type U. Ether bond formation, via a Mitsunobu-type reaction or from a corresponding alkyl halide, leads to a compound of type V. Reduction leads to an aldehyde of type W, then reductive amination to an amine of type X. An alcohol of type HO(CH₂)ᵥO(CH₂)_{w}R⁷ or HOCH₂CH(R⁶)CH₂R⁵ can be introduced in the same way. 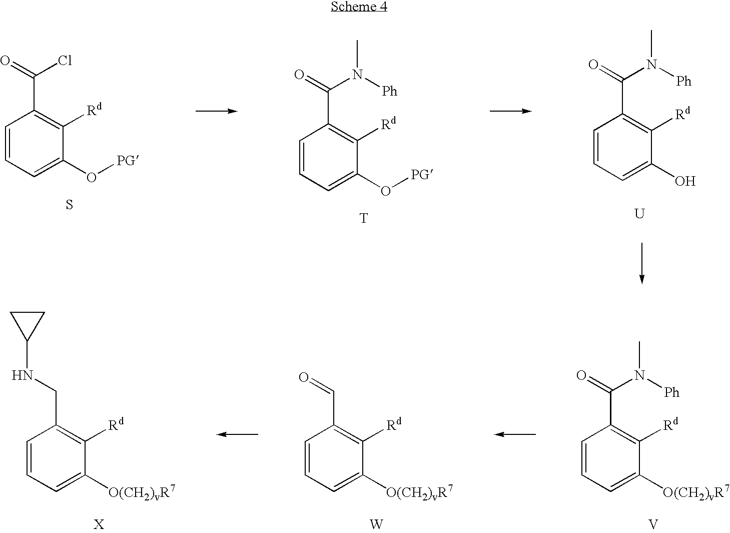
Preparation of Final Compounds

From precursors prepared as described above, the final compounds may be prepared using parallel chemistry techniques. For the specific examples, see the experimental part.

Diazabicyclononenes of type of H can be deprotected using standard procedures (Scheme 5). Purification by preparative HPLC might give the corresponding TFA salts or formate salts.

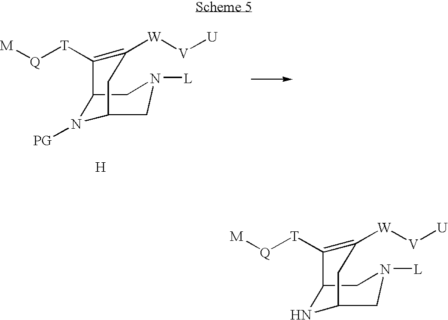

The following examples serve to illustrate the present invention in more detail. They are, however, not intended to limit its scope in any manner.

### EXAMPLES

Abbreviations

ACE Angiotensin Converting Enzyme

Ang Angiotensin

aq. aqueous

Boc tert-Butyloxycarbonyl

BSA Bovine serum albumine

BuLi n-Butyllithium

conc. concentrated

DIBAL Diisobutyl aluminium hydride

DIPEA Diisopropylethylamine

DMAP 4-N,N-Dimethylaminopyridine

DMF N,N-Dimethylformamide

DMSO Dimethylsulfoxide

EDC HCl Ethyl-N,N-dimethylaminopropylcarbodiimide hydrochloride

EIA Enzyme immunoassay

Et Ethyl

EtOAc Ethyl acetate

FC Flash Chromatography

HOBt Hydroxybenzotriazol

MeOH Methanol

org. organic

PG protecting group

RAS Renin Angiotensin System

rt room temperature

sat. saturated

sol. Solution

TBAF Tetra-n-butylammonium fluoride

TBDMS tert-Butyldimethylsilyl

Tf Trifluoromethylsulfonyl

THF Tetrahydrofuran

Preparation of the Precursors

### (rac.)-(1R*,5S*)-7-[4-(3-Hydroxypropyl)phenyl]-3,9-diazabicyclo[3.3.1]non-6-ene-3,6,9-tricarboxylic acid 3,9-di-tert-butyl ester 6-ethyl ester (D)

7-[4-(3-Hydroxypropyl)phenyl]-9-methyl-3,9-diazabicyclo[3.3.1]non-6-ene-3,6-dicarboxylic acid 3-tert-butyl ester 6-ethyl ester (patent application WO03/093267, 32.0 g, 57.3 mmol) was dissolved in dry 1,2-dichlorethane (590 mL). NaHCO₃ (48.2 g, 573 mmol) and 1-chloroethyl chloroformate (62.5 mL, 573 mmol) were added, and the suspension was heated to 80° C. After 3 h the reaction mixture was allowed to cool to rt. The mixture was filtered and the filtrate was evaporated under reduced pressure. The residue was dried 15 min under high vacuum. The product was then diluted in MeOH (400 mL), and the mixture was heated to 50° C. for 20 min. The reaction mixture was allowed to cool to rt, and the solvents were removed under reduced pressure. The yellow solid was dried under high vacuum for 1 h. The solid was dissolved in CH₂Cl₂ (190 mL), and the solution was cooled to 0° C. DIPEA (49.1 mL, 287 mmol), and Boc₂O (37.5 g, 172 mmol) were added. The reaction mixture was stirred overnight while warming up to rt. The reaction mixture was diluted with CH₂Cl₂ (110 mL). The organic layer was washed with aq. 1M HCl (2×300 mL), and aq. sat. NaHCO₃ (300 mL). The organic layer was dried over MgSO₄, filtered, and the solvents were evaporated under reduced pressure. Purification of the residue by FC(CH₂Cl₂/MeOH 100:0→2:98→5:95) yielded the title compound (26.2 g, 86%).
(rac.)-(1R*,5S*)-7-{4-[3-(2-Chloro-3,6-difluorophenoxy)propyl]phenyl}-3,9-diazabicyclo[3.3.1]non-6-ene-3,6,9-tricarboxylic acid 3,9-di-tert-butyl ester 6-ethyl ester (E)

A mixture of compound D (56.0 g, 106 mmol), 2-chloro-3,6-difluorophenol (34.8 g, 211 mmol), azadicarboxylic dipiperidide (53.4 g, 211 mmol) and PBu₃ (85%, 83 mL, 317 mmol) in toluene (1.20 L) was heated to reflux under nitrogen for 1 h. The mixture was allowed to cool to rt. The mixture was diluted with EtOAc (2.00 L), and the mixture was washed with aq. 1M NaOH (2×900 mL). The org. extracts were dried over MgSO4, filtered, and the solvents were removed under reduced pressure. Purification of the residue by FC (EtOAc/heptane 1:19→1:1) yielded the title compound (67.5 g, 94%).

### (rac.)-(1R*,5S*)-7-{4-[3-(2-Chloro-3,6-difluorophenoxy)propyl]phenyl}-3,9-diazabicyclo[3.3.1]non-6-ene-3,6,9-tricarboxylic acid 3,9-di-tert-butyl ester (F)

A mixture of compound E (67.5 g, 99.6 mmol) in aq. 1M NaOH (700 mL) and EtOH (1.40 L) was stirred at 80° C. overnight. The mixture was partially evaporated under reduced pressure, and EtOAc (500 mL) was added. The aq. phase was acidified with aq. 3M HCl, and the mixture was extracted. The org. layer was separated, dried over MgSO₄, filtered, and the solvents were removed under reduced pressure. The residue was dried under high vacuum, giving a 1:1 mixture of compounds 13 and 14, which was used further without purification (61.8 g, 95%).

### (rac.)-(1R*,5S*)-7-{4-[3-(2-Chloro-3,6-difluorophenoxy)propyl]phenyl}-6-{cyclopropyl-[2-(2-methoxyethoxy)-3-methylpyridin-4-ylmethyl]carbamoyl}-3,9-diazabicyclo[3.3.1]non-6-ene-3,9-dicarboxylic acid di-tert-butyl ester (G1)

A mixture of compound F (3.75 g, 5.77 mmol), amine R (3.08 g, 13.1 mmol), DIPEA (3.95 mL, 23.1 mmol), DMAP (177 mg, 1.44 mmol), HOBt (1.17 g, 8.65 mmol) and EDC.HCl (3.32 g, 17.3 mmol) in CH₂Cl₂ (60 mL) was stirred at rt for 3 days. The mixture was washed with aq. 1M HCl, and aq. sat. NaHCO₃. The org. extracts were dried over MgSO₄, filtered, and the solvents were removed under reduced pressure. Pzrification of the residue by FC (EtOAc/heptane 1:9→1:1) yielded the title compound (2.56 g, 51%).

### (rac.)-(1R*,5S*)-7-{4-[3-(2-Chloro-3,6-difluorophenoxy)propyl]phenyl}-6-{cyclopropyl-[3-(2-methoxyethoxy)-2-methylbenzyl]carbamoyl}-3,9-diazabicyclo[3.3.1]non-6-ene-3,9-dicarboxylic acid di-tert-butyl ester (G2)

A mixture of compound F (3.75 g, 5.77 mmol), amine X (3.08 g, 13.1 mmol), DIPEA (3.95 mL, 23.1 mmol), DMAP (177 mg, 1.44 mmol), HOBt (1.17 g, 8.65 mmol) and EDC.HCl (3.32 g, 17.3 mmol) in CH₂Cl₂ (60 mL) was stirred at rt for 3 days. The mixture was washed with aq. 1M HCl, and aq. sat. NaHCO₃. The org. extracts were dried over MgSO4, filtered, and the solvents were removed under reduced pressure. Pzrification of the residue by FC (EtOAc/heptane 1:9→1:1) yielded the title compound (3.15 g, 63%).

### 2-Chloro-N-phenylisonicotinamide (N)

To the sol. of 2-chloro-isonicotinoyl chloride (Anderson, W. K., Dean, D. C., Endo, T., J. Med. Chem., 1990, 33, 1667, 10 g, 56.8 mmol) in 1,2-dichloroethane (100 mL) was added at 0° C. a sol. of aniline (5.70 mL, 62.5 mmol) and DIPEA (10.2 ml, 59.6 mmol) in 1,2-dichloroethane (10 ml) during ca. 30 min. The reaction was stirred at 0° C. for ca. 30 min and subsequently for 1 h at 95° C. Water (30 mL) was added at rt and the mixture was filtered-off. The filtrate was extracted with CH₂Cl₂ (200 mL). The combined org. extracts were dried over MgSO₄, filtered, and the solvents were removed under reduced pressure. The residue was crystallized from MeOH/water 1:10 (110 mL), yielding the title compound (12.12 g, 92%). LC-MS: R_{T}=0.87 min; ES⁺=233.1.

### 2-Chloro-3-N-dimethyl-N-phenylisonicotinamide (O)

To a sol. of compound N (8.79 g, 37.8 mmol) in THF (90 mL) was added BuLi (1.6M in hexane, 52 mL, 83.2 mmol) at −78° C. After 30 min MeI (7.70 mL, 124 mmol) was added dropwise at the same temperature. The mixture was stirred at −78° C. for 1 h, and was warmed up to 33° C. The mixture was stirred at 33° C. for 30 min. Aq. 10% NH₄OH was added dropwise at rt, and the mixture was extracted with Et₂O. The org. extracts were dried over MgSO4, filtered, and the solvents were evaporated under reduced pressure. Purification by FC yielded the title compound (8.67 g, 88%). LC-MS:R_{T}=0.85 min; ES⁺=261.2.

### 2-Chloro-3-methylpyridine-4-carbaldehyde (P)

To the sol. of pyridine derivative 0 (9.58 g, 36.7 mmol) in CH₂Cl₂ (190 mL) was at −78° C. added DIBAL (1M in CH₂Cl₂, 55.1 mL, 55.1 mmol), and the mixture was stirred at −78° C. for 1.5 h. Aq. sat. tartaric acid monosodium monokalium salt in water (20 ml) was added and the mixture was allowed to warm up to rt. Water was added and the mixture was extracted with CH₂Cl₂. The org. extracts were dried over MgSO4, filtered, and the solvents were removed under reduced pressure. Purification of the residue by FC yielded the title compound (4.4 g, 77%). LC-MS:R_{T}=0.76 min; ES⁺=156.1.

### (2-Chloro-3-methylpyridin-4-ylmethyl)-cyclopropylamine (O)

A sol. of aldehyde P (4.70 g, 30.2 mmol) and cyclopropylamine (4.20 ml, 60.4 mmol) in MeOH (65 mL) was stirred at rt for 4 h. NaBH₄ (1.55 g, 39.2 mmol) was added and the mixture was stirred at rt for 12 h. Water and subsequently aq. 1M NaOH were added, and the solvents were partially removed under reduced pressure. The water phase was extracted with CH₂Cl₂ (2×). The combined org. extracts were dried over MgSO₄, filtered, and the solvents were removed under reduced pressure. Purification of the crude by FC yielded the title compound (4.66 g, 79%). LC-MS:R_{T}=0.43 min; ES⁺=197.1.

### Cyclopropyl-[2-(2-methoxyethoxy)-3-methylpyridin-4-ylmethyl]amine (R)

NaH (60% suspension, 8.76 g, 381 mmol) was added to 2-methoxyethanol (210 mL) over ca.2 h at rt. Subsequently, compound Q (15.0 g 76.3 mmol) was added, and the mixture was heated at 80° C. for 3 days. The mixture was allowed to cool to rt, and ice was added. The mixture was extracted with EtOAc (3×). The org. extracts were dried over MgSO₄, filtered, and the solvents were removed under reduced pressure. Purification of the residue by HPLC yielded the title compound (5.4 g, 30%).

### Acetic acid 2-methyl-3-(methylphenylcarbamoyl)phenyl ester (T)

DIPEA (26.6 mL, 155 mmol) was added to a sol. of N-methylanaline (12.4 mL, 114 mmol) in CH₂Cl₂ (200 mL), and the mixture was cooled to 0° C. A sol. of 3-acetoxy-2-methylbenzoyl chloride (22.0 g, 103 mmol) in CH₂Cl₂ (100 mL) was added dropwise. The mixture was stirred for 40 min at 0° C., and the mixture was washed with aq. sat. NH₄Cl. The org. extract were dried over MgSO₄, filtered, and the solvents were removed under reduced pressure. Purification by FC (EtOAc/heptane 1:4→1:3→1:2) yielded the title compound (30.3 g, quantitative).

### 3-Hydroxy-2,N-dimethyl-N-phenylbenzamide (U)

K₂CO₃ (40.0 g, 138 mmol) was added in portions to a sol. of compound T (27.3 g, 96.4 mmol) in MeOH (275 mL). The mixture was stirred for 1.5 h, and the solvents were removed under reduced pressure. The mixture was diluted with CHCl₃, and washed with aq. 1M HCl. The aq. phase was extracted back with CHCl₃. The combined org. extracts were washed with brine, dried over MgSO4, filtered, and the solvents were removed under reduced pressure. The title compound was used without further purification (23.1 g, 99%).

### 3-(2-Methoxyethoxy)-2,N-dimethyl-N-phenylbenzamide (V)

A mixture of compound U (23.1 g, 95.7 mmol) and K₂CO₃ (19.8 g, 144 mmol) in DMF (350 mL) was stirred for 1 h at rt. A sol. of 2-bromoethyl methyl ether (13.0 mL, 138 mmol) in DMF (20 mL) was added dropwise, and the mixture was stirred for 6 h at 110° C. The mixture was allowed to cool to rt, and the solvents were removed under reduced pressure. The residue was diluted with EtOAc, and washed with water and brine. The org. extracts were dried over MgSO₄, filtered, and the solvents were removed under reduced pressure. Purification of the residue by FC (EtOAc/heptane 1:1) yielded the title compound (25.9 g, 90%).

### 3-(2-Methoxyethoxy)-2-methylbenzaldehyde (W)

A sol. of compound V (23.9 g, 79.8 mmol) in THF (200 mL) was cooled to −78° C. DIBAL (1M in THF, 120 mL, 120 mmol) was added slowly while keeping the temperature below −70° C. The mixture was stirred at −78° C. for 3 h. Another portion of DIBAL (1M in THF, 64 mL, 64 mmol) was added again, and the mixture was stirred for 90 min. Aq. sodium potassium tartrate (104 g, 368 mmol in 200 mL water) was added, and the mixture was allowed to warm up to rt, and stirred for 3 days. The mixture was extracted with Et₂O (3×). The combined org. extracts were washed with brine, dried over MgSO₄, filtered, and the solvents were removed under reduced pressure. Purification by FC (EtOAc/heptane 1:4) yielded the title compound (2.7 g, 16%).

### Cyclopropyl-[3-(2-methoxyethoxy)-2-methylbenzyl]amine (X)

A mixture of compound W (2.70 g, 13.9 mmol) and cyclopropylamine (1.95 mL, 27.8 mmol) in MeOH (20 mL) was stirred at rt overnight. The mixture was cooled to 0° C. and NaBH₄ (0.68 g, 18.1 mmol) was added. The mixture was stirred for 1 h at 0° C. Aq. 1M NaOH (10 mL) was added, and the solvents were removed under reduced pressure. The residue was diluted with EtOAc and washed with aq. 1M NaOH. The org. extracts were dried over MgSO₄, filtered, and the solvents were removed under reduced pressure. Purification of the residue by FC yielded the title compound (2.84 g, 87%).

Preparation of the Final Compounds

### Example 1

### (rac.)-(1R*,5S*)-7-{4-[3-(2-Chloro-3,6-difluorophenoxy)propyl]phenyl}-3,9-diazabicyclo[3.3.1]non-6-ene-6-carboxylic acid cyclopropyl-[2-(2-methoxy-ethoxy)-3-methylpyridin-4-ylmethyl]amide

A sol. of compound G1 (2.56 g, 2.95 mmol) in CH₂Cl₂ (25 mL) was cooled to 0° C. HCl (4M in dioxane, 25 mL) was added. The mixture was stirred for 1 h at 0° C., and 1 h at rt. The solvents were rapidly removed under reduced pressure, and the residue was dried under high vacuum. the residue was diluted with CH₂Cl₂ and washed with aq. 1M NaOH (2×). The org. extracts were dried over MgSO₄, filtered, and the solvents were removed under reduced pressure. Purification of the residue by FC (MeOH/CH₂Cl₂ 5:95→7:93→1:9→12:88→15:85→20:80) yielded the title compound (1.55 g, 78%).

### Example 2

### (rac.)-(1R*,5S*)-7-{(4-[3-(2-Chloro-3,6-difluorophenoxy)propyl]phenyl}-3,9-diazabicyclo[3.3.1]non-6-ene-6-carboxylic acid cyclopropyl-[3-(2-methoxy-ethoxy)-2-methylbenzyl]amide

A sol. of compound G2 (3.15 g, 3.63 mmol) in CH₂Cl₂ (30 mL) was cooled to 0° C. HCl (4M in dioxane, 30 mL) was added. The mixture was stirred for 1 h at 0° C., and 1 h at rt. The solvents were rapidly removed under reduced pressure, and the residue was dried under high vacuum. the residue was diluted with CH₂Cl₂ and washed with aq. 1M NaOH (2×). The org. extracts were dried over MgSO4, filtered, and the solvents were removed under reduced pressure. Purification of the residue by FC (MeOH/CH₂Cl₂ 5:95→7:93→1:9→12:88→15:85→20:80) yielded the title compound (1.96 g, 81%).

The following assay was carried out in order to determine the activity of the compounds of general formula I and their salts.

Inhibition of Human Recombinant Renin by the Compounds of the Invention

The enzymatic in vitro assay was performed in 384-well polypropylene plates (Nunc). The assay buffer consisted of 10 mM PBS (Gibco BRL) including 1 mM EDTA and 0.1% BSA. The incubates were composed of 50 μL per well of an enzyme mix and 2.5 μL of renin inhibitors in DMSO. The enzyme mix was premixed at 4° C. and consists of the following components:
human recombinant renin (0.16 ng/mL)
synthetic human angiotensin(1-14) (0.5 μM)
hydroxyquinoline sulfate (1 mM)

The mixtures were then incubated at 37° C. for 3 h.

To determine the enzymatic activity and its inhibition, the accumulated Ang I was detected by an enzyme immunoassay (EIA) in 384-well plates (Nunc). 5 μL of the incubates or standards were transferred to immuno plates which were previously coated with a covalent complex of Ang I and bovine serum albumin (Ang 1-BSA). 75 μL of Ang I-antibodies in essaybuffer above including 0.01% Tween 20 were added and a primary incubation made at 4° C. overnight. The plates were washed 3 times with PBS including 0.01% Tween 20, and then incubated for 2 h at rt with an antirabbit-peroxidase coupled antibody (WA 934, Amersham). After washing the plates 3 times, the peroxidase substrate ABTS (2.2′-azino-di-(3-ethyl-benzthiazolinsulfonate), was added and the plates incubated for 60 min at room temperature. After stopping the reaction with 0.1 M citric acid pH 4.3 the plate was evaluated in a microplate reader at 405 nm. The percentage of inhibition was calculated of each concentration point and the concentration of renin inhibition was determined that inhibited the enzyme activity by 50% (IC₅₀). The IC₅₀-values of all compounds tested are below 100 nM. However selected compounds exhibit a very good bioavailibility and are metabolically more stable than prior art compounds.

Examples of Inhibition:

Example 1: 1.00 nM

Example 2: 1.05 nM

1. Compounds of the general formula I 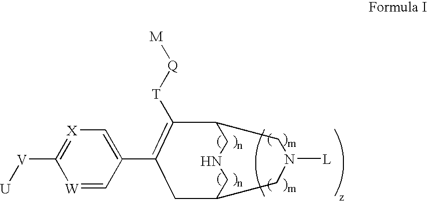
wherein
X and W represent independently a nitrogen atom or a —CH— group;
V represents —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; or —CH₂—CH₂-A-CH₂—CH₂—B—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH2)ₚN(R¹)SO₂—; or —COO—;
Q represents lower alkylene; lower alkenylene;
M represents aryl-O(CH₂)ᵥR⁷; heteroaryl-O(CH₂)ᵥR⁷; aryl-O(CH₂)ₙO(CH₂)_{w}R⁷; heteroaryl-(CH₂)ᵥO(CH₂)_{w}R⁷; aryl-OCH₂CH(R⁶)CH₂R⁵; heteroaryl-OCH₂CH(R⁶)CH₂R⁵;
L represents —R³; —COR³; —COOR³; —CONR²R³; —SO₂R³; —SO₂NR²R³; —COCH(Aryl)₂;
R¹ represents hydrogen; lower alkyl; lower alkenyl; lower alkinyl; cycloalkyl; aryl; cycloalkyl-lower alkyl;
R² and R2′ independently represent hydrogen; lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl;
cycloalkyl-lower alkyl; aryl-lower alkyl; heteroaryl-lower alkyl; heterocyclyl-lower alkyl;
aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, —NH(NH)NH₂, —NR⁴R⁴′ or lower alkyl, with the proviso that a carbon atom is attached at the most to one heteroatom in case this carbon atom is sp3-hybridized;
R⁴ and R⁴′ independently represents hydrogen; lower alkyl; cycloalkyl; cycloalkyl-lower alkyl;
hydroxy-lower alkyl; —COOR²; —CONH₂;
R⁵ represents —OH, lower alkoxy, —OCOR², —COOR², —NR²R²′, —OCONR²R²′, —NCONR²R²′, cyano, —CONR²R²′, SO₃H, —SONR²R²′, —CO-morpholin-4-yl, —CO—((4-loweralkyl)piperazin-1-yl), —NH(NH)NH₂, —NR⁴R⁴′, with the proviso that a carbon atom is attached at the most to one heteroatom in case this carbon atom is sp3-hybridized;
R⁶ represents —OH, OR²; OCOR²; OCOOR²; or R⁶ and R⁵ form together with the carbon atoms to which they are attached a 1,3-dioxolane ring which is substituted in position 2 with R² and R²′;
or R⁶ and R⁵ form together with the carbon atoms to which they are attached a 1,3-dioxolan-2-one ring;
R⁷ represents lower alkoxy;
m and n represent the integer 0 or 1, with the proviso that in case m represents the integer 1, n is the integer 0, and in case n represents the integer l, m is the integer 0;
p is the integer 1, 2, 3 or 4;
r is the integer 3, 4, 5, or 6;
s is the integer 2, 3, 4, or 5;
t is the integer 1, 2, 3, or 4;
u is the integer 1, 2, or 3;
v is the integer 1, 2, 3, or 4;
w is the integer 1 or 2;
z is the integer 0 or 1;
in any form, including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

2. Compounds of general formula I according to claim 1 wherein X, W, V, U, T, Q, L, and M are as defined in general formula I and
zis 1
n is 0
m is 1,
in any form, including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

3. Compounds of general formula I according to claim 1 wherein X, W, V, U, T, Q, M, m, and n are as defined in general formula I and
z is 1
L represents —COR³″; —COOR³″; —CONR²″R³″;
R²″ and R³″ represent independently lower alkyl; lower cycloalkyl-lower alkyl, which lower alkyl and lower cycloalkyl-lower alkyl are undubstituted or mono-substituted with halogen, —CN, —OH, —OCOCH₃, —CONH₂, —COOH, or —NH₂, with the proviso that a carbon atom is attached at the most to one heteroatom in case this carbon atom is sp3-hybridized,
in any form, including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

4. Compounds of general formula I according to claim 1 wherein X, W, V, U, L, m, n, and z are as defined in general formula I and
T represents —CONR¹—;
Q represents methylene;
M represents aryl-O(CH₂)ᵥR⁷; heteroaryl-O(CH₂)ᵥR⁷; aryl-OCH₂CH(R⁶)CH₂R⁵; heteroaryl-OCH₂CH(R⁶)CH₂R⁵;
in any form, including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

5. Compounds of general formula I according to claim 1 wherein X, W, U, L, T, Q, M, m, n, and z are as defined in general formula I and
V represents —CH₂CH₂O—; —CH₂CH₂CH₂O—; —OCH₂CH₂O—;
in any form, including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

6. Compounds of general formula I according to claim 1 wherein V, U, T, Q, M, L, m, n, and z are as defined in general formula I and
X and W represent a —CH— group
in any form including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

7. Compounds of general formula I according to claim 1 wherein X, W, V, Q, T, M, L, m, n, and z are as defined in general formula I and
U is a mono-, di-, or trisubstituted phenyl whereby the substituents are halogen; lower alkyl or lower alkoxy
in any form including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

8. Compounds according to claim 1 of general formula I, wherein
X and W represent a —CH— group;
V represents -A-(CH₂)s-;
A represents —O—;
U represents phenyl, trisubstituted with halogen;
T represents —CONR¹—;
Q represents C1-C4 alkyl;
M represents phenyl —O— (CH₂)v R⁷ or pyridyl-O— (CH₂)v R⁷;
L represents R³;
R¹ represents cycloalkyl;
R³ represents hydrogen, C1-C4 alkyl;
R⁷ represents C1-C4 alkoxy;
m represents the integer 1;
n represents the integer 0;
z represents the integer 1;
s represents the integer 3;
v represents the integer 2;
in any form, including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

9. Compounds according to claim 1 of general formula I, wherein
X and W represent a —CH— group;
V represents —O—CH₂—CH₂—CH₂—;
U represents phenyl, trisubstituted independently with Fluoro and Chloro;
T represents —CONR¹—;
Q represents —CH₂—;
M represents phenyl —O— (CH₂)v R⁷ or pyridyl-O— (CH₂)v R⁷;
L represents R³;
R¹ represents cyclopropyl;
R³ represents hydrogen;
R⁷ represents methoxy;
m represents the integer 1;
n represents the integer 0;
z represents the integer 1;
z represents the integer 3;
v represents the integer 2;
in any form, including optically pure enantiomers, mixtures of enantiomers such as racemates, diastereomers, mixtures of diastereomers, diastereomeric racemates, mixtures of diastereomeric racemates, and the meso-form; as well as free or pharmaceutically acceptable salts, solvent complexes and morphological forms.

10. The compounds according to claim 1 selected from the group consisting of
(rac.)-(1R*,5S*)-7-{4-[3-(2-chloro-3,6-difluorophenoxy)propyl]phenyl}-3,9-diazabicyclo[3.3.1]non-6-ene-6-carboxylic acid cyclopropyl-[2-(2-methoxy-ethoxy)-3-methylpyridin-4-ylmethyl]amide, and
(rac.)-(1R*,5S*)-7-{4-[3-(2-chloro-3,6-difluorophenoxy)propyl]phenyl}-3,9-diazabicyclo[3.3.1]non-6-ene-6-carboxylic acid cyclopropyl-[3-(2-methoxy-ethoxy)-2-methylbenzyl]amide.

11. Pharmaceutical compositions comprising a compound of claim 1 in combination or association with a pharmaceutically acceptable diluent or carrier.

12. A method for the treatment or prophylaxis of diseases which are related to the RAS comprising hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, complications of treatment with immunosuppressive agents after organ transplantation, and other diseases which are related to the RAS, which method comprises administering an effective amount of a compound according to claim 1 to a human being or animal.

13. (canceled)

14. The method according to claim 12 further comprising administering an effective amount of a pharmacologically active compound selected from ACE inhibitors, angiotensin II receptor antagonists, endothelin receptor antagonists, vasodilators, calcium antagonists, potassium activators, diuretics, sympatholitics, beta-adrenergic antagonists, and alpha-adrenergic antagonists.

15. A compound according to claim 1 which is (rac.)-(1R*,5S*)-7-{4-[3-(2-chloro-3,6-difluorophenoxy)propyl]phenyl}-3,9-diazabicyclo[3.3.1]non-6-ene-6-carboxylic acid cyclopropyl-[2-(2-methoxy-ethoxy)-3-methylpyridin-4-ylmethyl]amide, or an optically pure enantiomer thereof, in free or pharmaceutically acceptable salt form.

16. A compound according to claim 1 which is (rac.)-(1R*,5S*)-7-{4-[3-(2-chloro-3,6-difluorophenoxy)propyl]phenyl}-3,9-diazabicyclo[3.3.1]non-6-ene-6-carboxylic acid cyclopropyl-[3-(2-methoxy-ethoxy)-2-methylbenzyl]amide, or an optically pure enantiomer thereof, in free or pharmaceutically acceptable salt form.
